(19) United States
(12) Patent Application Publication
(10) Pub. No.: US 2021/0299406 A1
(43) Pub. Date: Sep. 30, 2021

(54) SYSTEM, DEVICE, AND METHOD FOR VASCULAR ACCESS

(71) Applicant: THE REGENTS OF THE UNIVERSITY OF COLORADO, A BODY CORPORATE, Denver, CO (US)

(72) Inventors: Juan-Pablo Idrovo, Denver, CO (US); Andrew Scallon, Aurora, CO (US)

(73) Assignee: THE REGENTS OF THE UNIVERSITY OF COLORADO, A BODY CORPORATE, Denver, CO (US)

(21) Appl. No.: 17/266,513

(22) PCT Filed: Aug. 6, 2019
(86) PCT No.: PCT/US19/45282

§ 371 Date: Feb. 5, 2021

Related U.S. Application Data
(60) Provisional application No. 62/715,135, filed on Aug. 6, 2018.

(51) Int. Cl. A61M 25/01; A61M 25/09; A61M 25/06

(52) U.S. Cl. CPC A61M 25/0136; A61M 25/09; A61M 2025/09008; A61M 2205/3327; A61M 25/06

(57) ABSTRACT

A vascular access system and device. The vascular access device includes a frame having a handle, a linear actuation assembly, and a needle retainer, according to various embodiments. The linear actuation assembly is mounted to the frame and has a translating portion configured to translate relative to the frame, according to various embodiments. The needle retainer is coupled to the translating portion and is configured to retainer a needle, according to various embodiments. In various embodiments, the needle retainer is one needle retainer of a plurality of needle retainers, the plurality of needle retainers is configured to retain a respective plurality of needles, and the plurality of needle retainers is configured to operably translate as a unit with the translating portion relative to the frame.

### CROSS-REFERENCE TO RELATED APPLICATIONS

This application claims priority to U.S. Provisional Patent Application Ser. No. 62/715,135, filed on Aug. 6, 2018, the entire contents of which are incorporated herein by reference in their entirety.

### FIELD

The present disclosure relates to vascular access devices, systems, and methods.

### BACKGROUND

Modern medicine often requires access to vascular vessels, such as venous or arterial vascular spaces (e.g., veins or arteries), to diagnose, monitor, or deliver treatment to a patient. For example, patients who present with inflammatory or infective processes, or patients who require active resuscitation, blood sampling, vital sign monitoring, or intravenous medications, may benefit from vascular access in treating or overcoming their condition. Each one of these interventions requires vascular access. However, factors such as the body habitus, anatomy, age, or current pathology of the patient may make vascular access challenging, even for the experienced surgeon. Moreover, critically ill patients may require central venous access in combination with arterial access.

To control bleeding in hemorrhagic shock situations, extremely invasive methods of aortic cross clamping, such as thoracotomies or laparotomies, traditionally have been employed. Resuscitative Endovascular Balloon Occlusion of the Aorta (“REBOA”), which involves deploying an occlusion balloon in the femoral artery for control of bleeding, has increased in popularity due to being less invasive. However, locating and accessing the femoral artery, especially in emergency settings, can be extremely challenging. Factors such as hypovolemia and the ongoing cardiopulmonary resuscitation may make the femoral artery almost invisible, even with guidance from a conventional ultrasound. Conventional techniques also are far from ergonomic. Therefore, whereas the REBOA technique clearly shows promise to be a lifesaving new procedure, vascular access limitations generally hinder its efficacy. Currently, there is a misfortunate 42% failure rate in percutaneously cannulating the femoral artery in trauma patients, which often results in conversion to operative groin cut-down. Thus, there is a need for improved vascular access as provided by the present disclosure.

### SUMMARY

In various embodiments, the present disclosure provides a vascular access device. The vascular access device includes a frame having a handle, a linear actuation assembly, and a needle retainer, according to various embodiments. The linear actuation assembly is mounted to the frame and has a translating portion configured to translate relative to the frame, according to various embodiments. The needle retainer is coupled to the translating portion and is configured to retainer a needle, according to various embodiments.

In various embodiments, the needle retainer is one needle retainer of a plurality of needle retainers, the plurality of needle retainers is configured to retain a respective plurality of needles, and the plurality of needle retainers is configured to operably translate as a unit with the translating portion relative to the frame. In various embodiments, the linear actuation assembly comprises a track along which the translation portion is configured to move. The frame may comprise a bottom surface configured to be in direct contact with a body of a patient, and the track may be oblique relative to the bottom surface of the frame. The needle retainer may be configured to retain the needle substantially parallel to the track.

In various embodiments, the handle is a first handle, the first handle extends above and is substantially perpendicular to the bottom surface of the frame, and the frame further comprises a second handle disposed between the track and the bottom surface of the frame. In various embodiments, the frame comprises a forward lip portion, wherein the forward lip portion is bent upwards relative to the bottom surface of the frame. The minor angle between the forward lip portion and a plane of the bottom surface is between 10 degrees and 25 degrees. In various embodiments, the linear actuation assembly comprises a rack and gear assembly and an actuation trigger. The frame may also define a compartment configured to house a transducer of a vascular vessel position sensor.

Also disclosed herein, according to various embodiments, is a vascular access system. The vascular access system may include a vascular access device and a vascular vessel position sensor. The vascular access device may include a frame and a needle retainer, wherein the needle retainer is movably mounted to the frame such that the needle retainer is configured to translate relative to the frame. The vascular vessel position sensor may include a transducer mounted to the frame.

In various embodiments, the needle retainer is one needle retainer of a plurality of needle retainers that is configured to translate as a unit relative to the frame and the vascular access system further comprises a plurality of needles respectively retained by the plurality of needle retainers. Each needle of the plurality of needles comprises at least one of a transparent tube and translucent tube at a back end of the needle, according to various embodiments. The system may further include a guide wire, a catheter, and an occlusion balloon. The guide wire may be configured to be inserted through a first needle of the plurality of needles in response to a tip of the first needle being positioned within a lumen defined by a vascular vessel of a patient. The catheter may be configured to be inserted along the guide wire into the lumen of the vascular vessel of the patient. The occlusion balloon may be configured to be deployed through the catheter into the lumen of the vascular vessel of the patient. In various embodiments, the frame of the vascular access device defines a compartment and the transducer of the vascular vessel position sensor is housed within the compartment.

Also disclosed herein, according to various embodiments, is a method of accessing a vascular vessel of a patient. The method includes engaging a bottom surface of a frame of a vascular access device against the patient, according to various embodiments. The method also includes moving the vascular access device relative to the patient to position the vascular access device in a desired position in proximity to the vascular vessel of the patient, according to various embodiments. Still further, the method includes, in response to the vascular device being in the desired position, advancing a needle retained by a needle retainer translatably mounted to the frame of the vascular access device, according to various embodiments.

In various embodiments, the needle is one needle of a plurality of needles, the needle retainer is one needle retainer of a plurality of needle retainers, and advancing the needle comprises collectively advancing the plurality of needles as a unit into the patient. In various embodiments, moving the vascular access device comprises utilizing feedback from a vascular vessel position sensor mounted to the frame of the vascular access device to ascertain the desired position. In various embodiments, the method further includes identifying a first needle of the plurality of needles that has its tip positioned within a lumen of the vascular vessel. The method may also include inserting a guide wire through the first needle, inserting a catheter along the guide wire into the lumen of the vascular vessel of the patient, and deploying an occlusion balloon through the catheter into the lumen of the vascular vessel of the patient.

The forgoing features and elements may be combined in various combinations without exclusivity, unless otherwise expressly indicated herein. These features and elements, as well as the operation of the disclosed embodiments, will become more apparent in light of the following description and accompanying drawings.

### BRIEF DESCRIPTION OF THE DRAWINGS

FIG. 1 is a perspective view of a vascular access system in an exemplary use position, in accordance with various embodiments;

FIG. 2 is a perspective view of a vascular access device, in accordance with various embodiments;

FIG. 3 is another perspective view of a vascular access device, in accordance with various embodiments;

FIG. 4A is a partial cross-sectional view of a vascular access device showing a linear actuation assembly and a plurality of needle retainers, in accordance with various embodiments;

FIG. 4B is a partial cross-sectional view of a vascular access device showing a linear actuation assembly and a single needle retainer, in accordance with various embodiments;

FIG. 5 is a cross-sectional side view of a handle of a vascular access device showing various exemplary angles of the vascular access device, in accordance with various embodiments;

FIG. 6 is a front view of a plurality of needle retainers of a vascular access device, in accordance with various embodiments;

FIG. 7 is a top view of a forward lip portion of a vascular access device showing a plurality of needles inserted into a patient, in accordance with various embodiments;

FIG. 8 is a schematic flow chart diagram of a method of accessing a vascular vessel, in accordance with various embodiments; and

FIG. 9 depicts anatomy of femoral and popliteal arteries, in accordance with various embodiments.

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

### DETAILED DESCRIPTION

The detailed description of exemplary embodiments herein makes reference to the accompanying drawings, which show exemplary embodiments by way of illustration. Although these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and is not limiting.

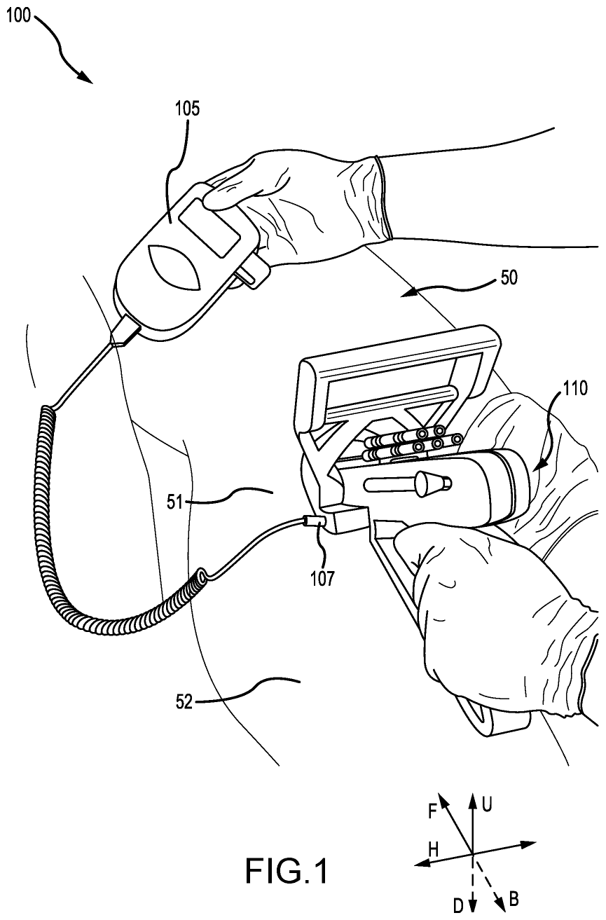

Disclosed herein, according to various embodiments and with reference to FIG. 1, is a vascular access system 100 that includes a vascular access device 110. The vascular access system 100 may also include a vascular vessel position sensor 105. Generally, the vascular access system 100, as described in greater detail below, enables a practitioner to rapidly and efficiently cannulate a vascular vessel, such as an artery or a vein, of a patient 50. For example, components of the vascular access system 100 may be utilized to perform a Resuscitative Endovascular Balloon Occlusion of the Aorta (“REBOA”), which involves deploying an occlusion balloon in the femoral artery for control of bleeding.

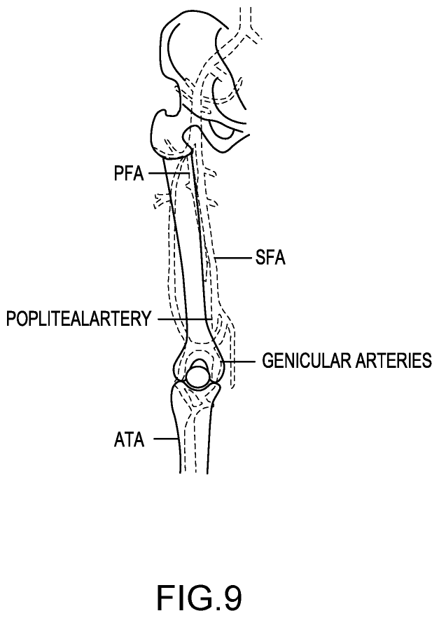

In various embodiments, the vascular access system 100 may utilize feedback from the vascular vessel position sensor 105 to position the vascular access device 110 in a desired position (e.g., as close as possible) relative to the vascular vessel of the patient to facilitate insertion of the one or more needles coupled to the vascular access device. In various embodiments, the vascular access device 110 includes one or more needle retainers configured to hold/retain a respective one or more needles. The one or more needles may be advanced into the patient until a needle pierces the desired vascular vessel and has its tip positioned within the lumen of the vascular vessel. Additional details pertaining to the system 100, device 110, and the associated method(s) of use are described in greater detail below.

Whereas the disclosed system 100 may be utilized for various procedures, one exemplary (e.g., non-limiting) procedure is cannulation of the femoral artery. For example, as shown in FIG. 1, the vascular access device 110 may be positioned adjacent the groin area 51 of the patient and may be generally be engaged against an upper leg/thigh 52 of the patient for cannulation of the femoral artery. Accordingly, the vascular access system 100 may facilitate the placement of a Resuscitative Endovascular Balloon Occlusion of the Aorta (“REBOA”). With momentary reference to FIG. 9, pertinent anatomy of femoral and popliteal arteries is provided. The common femoral artery (which may be 4-6 centimeters long) lies superficially in the groin and divides into the superficial femoral artery (“SFA”) and the popliteal femoral artery (“PFA”). The SFA extends down the medial thigh and passes through the adductor hiatus and the PFA commences below the adductor hiatus and passes vertically through popliteal fossa and divides to the tibioperoneal trunk and the anterior tibial artery. The common femoral artery for an adult generally has a diameter of 7-9 millimeters, with the SF having a diameter of 6-8 millimeters and the PFA having a diameter of 4-6 millimeters.

In various embodiments, and with continued reference to FIG. 1, the terms “front,” “forward,” or the like refer to elements, sections, orientations, directions, etc., of the vascular access device 110 that are situated or directed toward the upper body of the patient 50 in FIG. 1, whereas the terms “back,”, “backward,” or the like refer to elements, sections, orientations directions, etc. of the vascular access device 110 that are situated or directed toward the lower body of the patient 50 in FIG. 1. Further, according to various embodiments and with reference to FIG. 1, the terms “up,” “top,” “above,” “upward,” of the like refer to elements, sections, orientations, directions, etc., of the vascular access device 110 that are upward and away from the body of the patient 50 in FIG. 1, whereas the terms “down,” “bottom,” “below,” “downward,” or the like refer to elements, sections, orientations, directions, etc., of the vascular access device 110 that are situated or directed toward the body of the patient 50 in FIG. 1. Thus, axis F refers to the front/forward direction, axis B refers to the back/backward direction, axis U refers to the up/top direction, and axis D refers to the down/bottom direction of the vascular access device 110. Axis H refers generally to a horizontal direction.

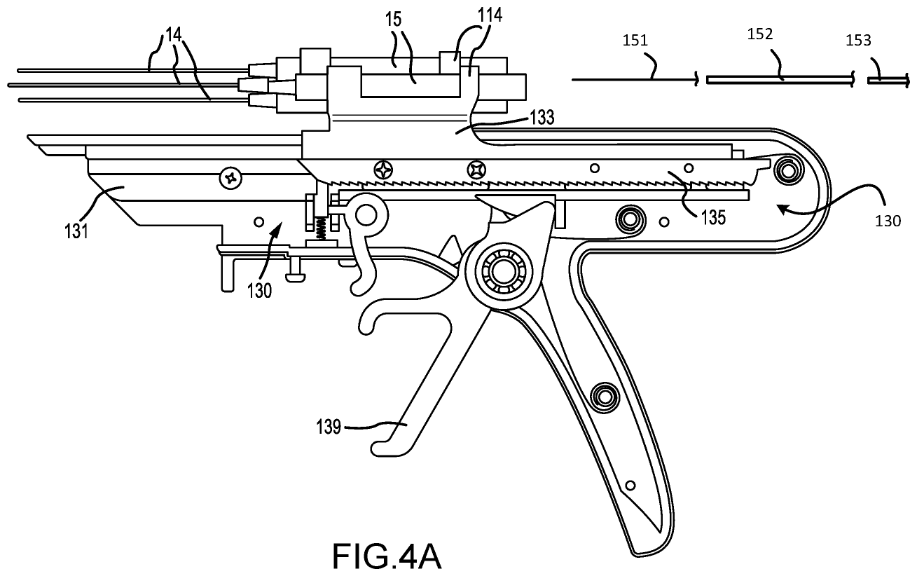

In various embodiments, and with reference to FIGS. 1-3, the vascular access device 110 has a frame 112 that includes one or more needle retainers. For example, the vascular access device 110 may include a single needle retainer 114B (FIG. 4B) for retaining/holding a single needle 14B (FIG. 4B) or the vascular access device may include a plurality of needle retainers 114 (FIGS. 1-3 and 4A) for retaining/holding a respective plurality of needles 14 (FIGS. 1-3 and 4A). Each needle may be mounted, connected, fastened or otherwise attached to each needle retainer 114. Though many of the embodiments disclosed herein reference “a plurality of needle retainers,” these embodiments may be implemented with a single needle retainer. However, in various embodiments a device having a plurality of needle retainers may have various advantages, specifically pertaining to locating the desired vascular vessel, as described in greater detail below and in accordance with various embodiments.

In various embodiments, the one or more needle retainers 114 are movably mounted to the frame 112, according to various embodiments. For example, and as described in greater detail below with reference to FIG. 4A, the vascular access device 110 may have a linear actuation assembly 130 that is controllable to translate the plurality of needle retainers 114 such that the plurality of needle retainers 114 move/translate collectively as a unit. Accordingly, the needles 14 held/retained by the plurality of needle retainers 114 may be advanced collectively to pierce the skin of the patient 50 until at least one of the tips of the needles held by the needle retainers 114 is positioned within a lumen of the vascular vessel (e.g., femoral artery). Thus, a practitioner may determine which needle of the plurality of needles 14 has entered the desired artery based on which needle has blood flow.

In various embodiments, the vascular vessel position sensor 105 includes a transducer 107 mounted to the frame 112. That is, the frame 112 may define a compartment 117 and the transducer 107 of the vascular vessel position sensor 105 may be retained within the compartment 117. In various embodiments, the vascular vessel position sensor may be an ultrasound probe or other such imaging device that may be mounted or otherwise attached to the frame 112. For example, the frame 112 may include a mounting interface and a sensing probe may be coupled thereto. In various embodiments, the vascular vessel position sensor 105 is configured to detect a position/location of the vascular vessel of the patient 50 and provide feedback to the practitioner pertaining to its position/location. For example, the vascular vessel position sensor 105 may provide audible or visible feedback to the practitioner, indicating when the transducer 107 is in close proximity to the vascular vessel, thereby enabling the practitioner to hone in on the target site for vascular access and correspondingly position the vascular access device 110 in a desired position relative to the body of the patient 50. In various embodiments, the vascular vessel position sensor 105 may be a Doppler or ultrasound device. A Doppler or ultrasound device may be especially well-suited for vascular detection.

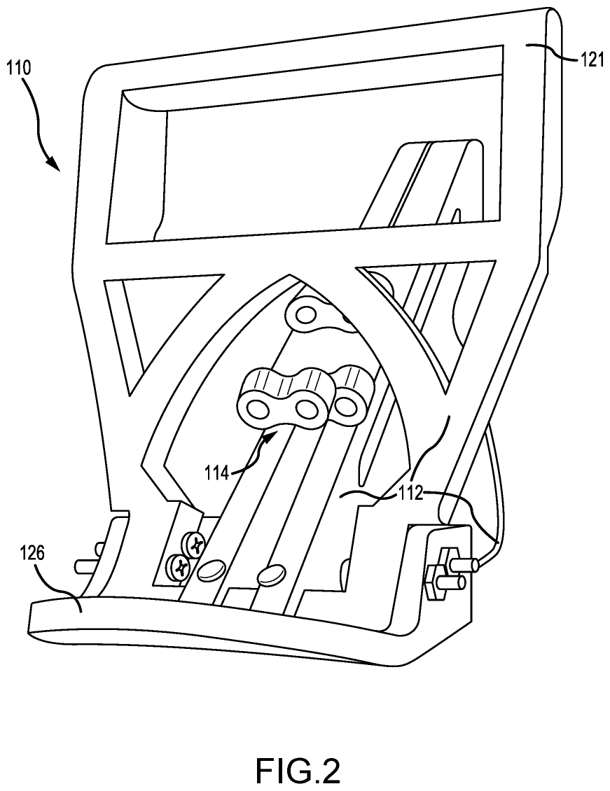

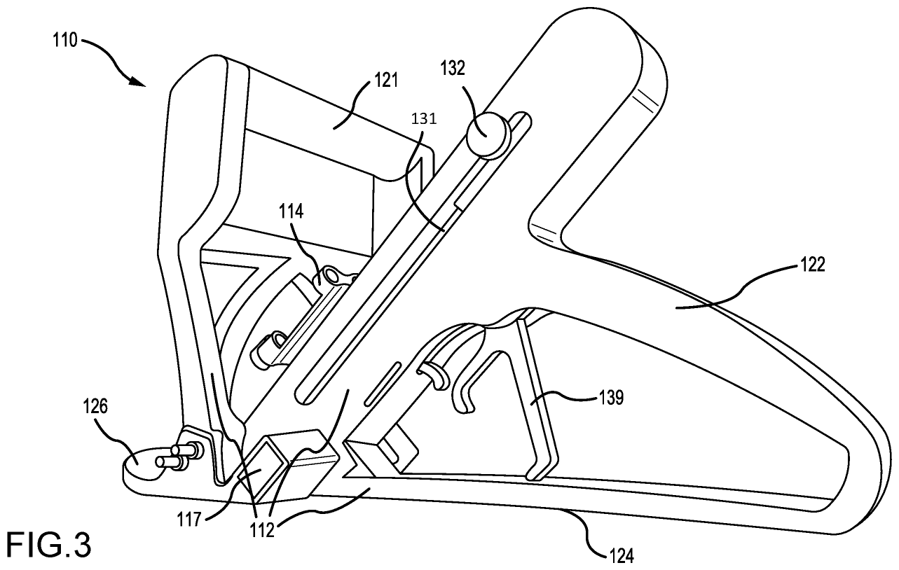

In various embodiments, and with reference to FIGS. 2 and 3, the frame 112 of the vascular access device 110 may have a handle 121. The handle 121 may be a first handle and the vascular access device 110 may further include a second handle 122. The first handle 121 may be situated substantially above the plurality of needle retainers 114 and the second handle 122 may be a portion of the frame 112 between the track 131 of the linear actuation assembly 130 (FIGS. 4A and 4B) and a bottom surface 124 of the frame 112. That is, the frame 112 may have a bottom surface 124 that is generally configured to be directly engaged against the body of the patient 50, and the second handle 122 may be the portion of the frame that extends between this bottom surface 124 and the track 131 along which the plurality of needle retainers 114 (with needles 14) are configured to translate.

This second handle 122 may define a void within which a trigger (e.g., an actuation trigger) 139 or other actuation mechanism of the linear actuation assembly 130 (FIGS. 4A and 4B) may extend, and thus the practitioner may be able to both grasp the second handle 122 and actuate the linear actuation assembly 130 via the trigger 139 using a single hand. The practitioner's other hand may grasp the first handle 121 to support and otherwise stabilize the device 110. In various embodiments, the linear actuation assembly also include a knob 132 of other feature for manually advancing the needle retainer(s) 114 over larger distances faster than the trigger 139. That is, the practitioner may use the knob 132 to approximate or retract the needle retainer(s) 114 from the patient, and may use the trigger 139 for the final precise and incremental advancement of the needle retainer(s) 114 (and thus the needles) into the vascular vessel of the patient.

In various embodiments, the frame 112 of the vascular access device 110 further includes a forward lip portion 126. The forward lip portion 126 may be bent upwards relative to the bottom surface 124 of the frame 112 and may further facilitate stabilization of the vascular access device 110. In various embodiments, the forward lip portion 126 may be especially pertinent for femoral artery access, as the forward lip portion 126 with its upturned orientation may engage skin and subcutaneous tissue from the proximal-groin/distal-abdomen of the patient 50 to facilitate positioning the device 110 in the correct position.

In various embodiments, and with reference to FIGS. 4A and 4B, an example of a linear actuation assembly 130 is provided. The linear actuation assembly 130 may include a linear track 131 and a translating portion 133 (e.g., a carriage) to which the plurality of needle retainers 114 are mounted. A linear bearing may be included to facilitate movement of the translating portion 133 along the track 131. In various embodiments, the linear actuation assembly 130 may be a rack and gear assembly, and thus may include a linear gear 135 (e.g., a rack) coupled to the translating portion 133 and configured to interface with the trigger 139 of the linear actuation assembly 130. That is, a tooth coupled to the trigger 139 may engage the linear gear 135 and actuate the translation. Reversion of the assembly may be prevented by a second tooth assembly connected to a release trigger. The activation of the release trigger may redact the assembly to its starting position, withdrawing the plurality of needles from the patient.

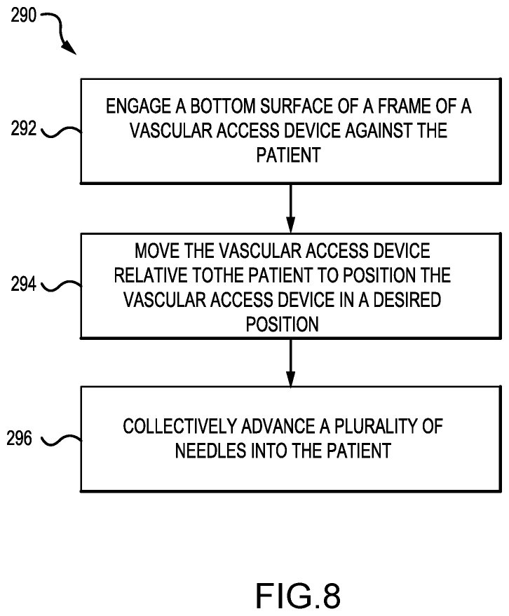

As shown in FIG. 4A, the needles 14 may have a transparent tube 15 or a translucent tube at a back end of the needle 14, and this transparent compartment may be configured to retain blood in response to the tip of at least one of the needles 14 entering the lumen of the vascular vessel. In various embodiments, as described in greater detail below with reference to the method of FIG. 8, a guide wire 151, a catheter 152, and/or an occlusion balloon 153 (shown schematically in FIG. 4A) may be sequentially inserted/deployed into the lumen of the vascular vessel. As mentioned above, the vascular access device may have a plurality of needle retainers 114 (FIG. 4A) coupled to the translating portion 133 of the linear actuation assembly 130 or the vascular access device may have a single needle retainer 114B (FIG. 4B) coupled to the translating portion 133 of the linear actuation assembly 130.

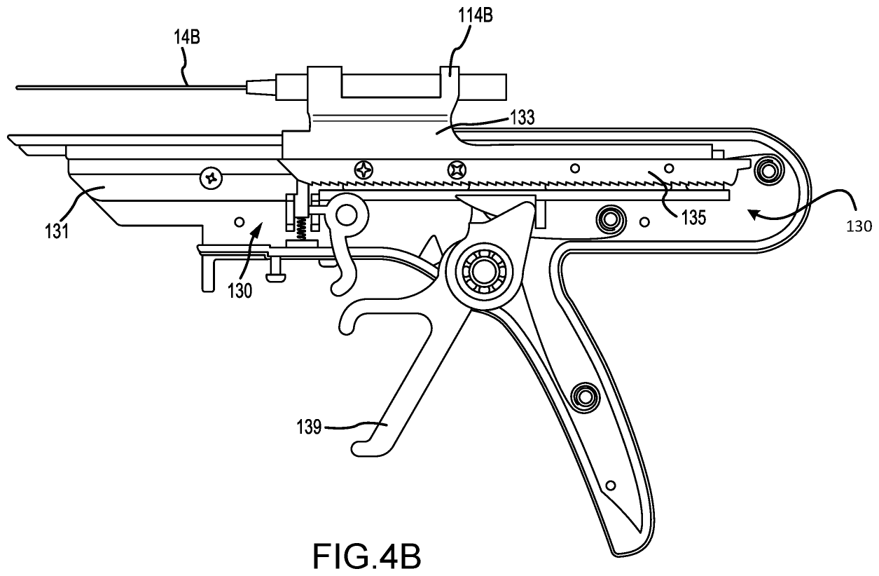

In various embodiments, and with reference to FIG. 5, a cross-sectional view of a front/forward portion of the vascular access device 110 is provided. In various embodiments, the first handle 121 may extend substantially perpendicular to the bottom surface 124 of the frame 112 of the vascular access device 110. As used in this context only, the term “substantially” means plus or minus 10 degrees. In various embodiments, an angle (i.e., the minor angle) between the forward lip portion 126 and a plane of the bottom surface 124 (i.e., the plane in which the bottom surface 124 extends) is between about 10 degrees and about 25 degrees. In various embodiments, this minor angle between the forward lip portion 126 and the plane of the bottom surface 124 is about 15 degrees. As used in this context only, the term “about” refers to plus or minus 2.5 degrees.

In various embodiments, FIG. 5 also shows a projection 141 of the linear track 131 along which the translating portion 133 of the linear actuation assembly 130 is configured to translate. This projection 141 generally shows the position of the linear track 131, and FIG. 5 shows an angle between the bottom surface 124 of the frame 112 of the vascular access device 110 and the linear track 131. That is, the track 131/141 may be oblique relative to the bottom surface 124 of the frame 112. In various embodiments, this angle is between about 30 degrees about 60 degrees. In various embodiments, this angle is about 45 degrees. As used in this context only, the term “about” refers to plus or minus 2.5 degrees. In various embodiments, and with momentary reference to FIG. 4A, the needles 14 may be held in the needle retainers 114 such that the needles 14 extend substantially parallel to the track 131 and thus extend parallel to the direction of the translation motion.

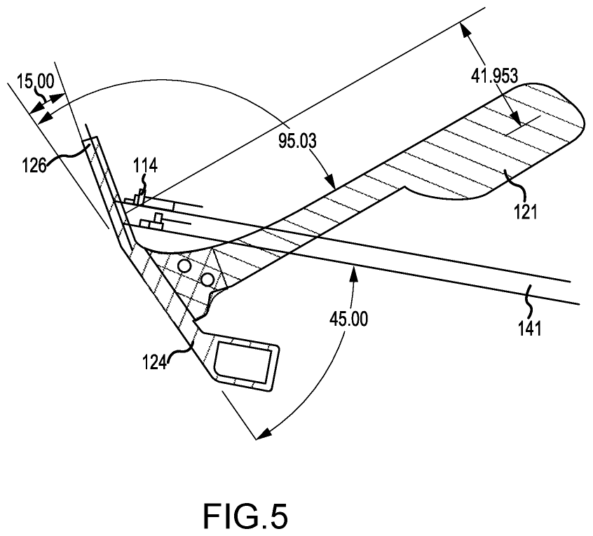

In various embodiments, and with reference to FIG. 6, the vascular access device may include, for example, one to five needle retainer(s). In various embodiments, the needle retainers 114 may not be in a linear configuration, and thus the needle retainers 114 may be offset as shown in FIG. 6. In such a configuration, the horizontal distance 143 between adjacent needle retainer slots may still be constant/uniform while also being shorter than would otherwise be possible if the needle retainer slots were side-by-side in a linear configuration. That is, distance 143 may be less than a diameter of the needle retainer slots.

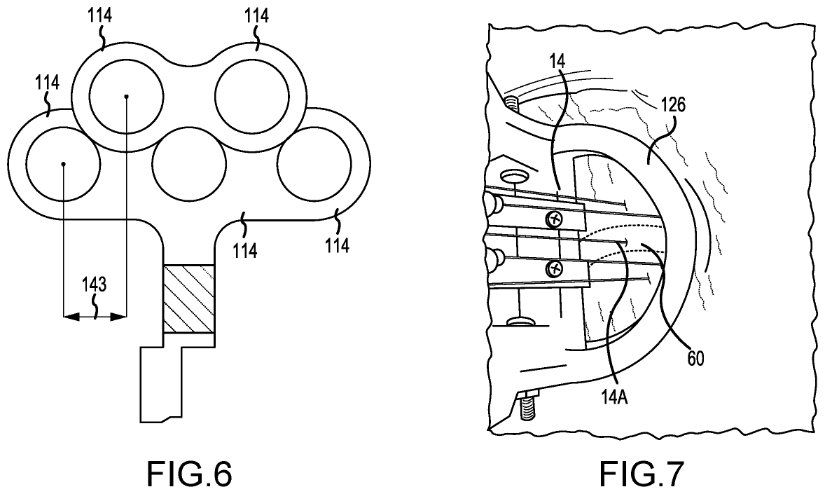

In various embodiments, and with reference to FIG. 7, a plurality of needles 14 are shown extending into the body of the patient 50, with a tip of a first needle 14A being positioned within a vascular vessel 60. As described above, the vascular vessel position sensor 105 may be configured to help locate the device in the general vicinity (e.g., over top of) the desired vascular vessel. The advancement of the multiple needles as a collective unit helps ensure that at least one of the needles will reach the desired vascular vessel, even though the other may not, thus enabling the device/system to be implemented without the vascular vessel position sensor 105.

FIG. 7 shows the patient having an open incision with features holding the skin and tissue of the patient open, but these are only included for the purposes of clearly showing the desired vascular vessel 60, and thus in actual practice/use of the system/device, the vascular vessel 60 is unlikely to be visible and it is unlikely there will be such an open cut-down/incision. Also shown in FIG. 7, the forward lip portion 126 of the frame 112 may define a window through which the needles 14 are configured to extend.

In various embodiments, the device may include a horizontal actuation assembly that is configured to adjust a horizontal position of the needles. That is, horizontal actuation assembly may be configured to enable motion of the one or more needle retainers in a direction perpendicular to the linear translation motion of the linear actuation assembly. However, in various embodiments, this horizontal adjustment is merely accomplished by moving the entire device 110 so that it is better situated above the desired vascular vessel.

In various embodiments, and with reference to FIG. 8, a method 290 of accessing a vascular vessel of a patient is provided. The method 290 may include engaging a bottom surface of a frame of a vascular access device against the patient at step 292. The method 290 may further include moving the vascular access device relative to the patient to position the vascular access device in a desired position in proximity to the vascular vessel of the patient at step 294. Still further, the method 290 may include, in response to step 294 (e.g., in response to the vascular device being in the desired position), collectively advancing a plurality of needles into the patient at step 296. The plurality of needles may be retained by a respective plurality of needle retainers that are translatably mounted relative to the frame of the vascular access device.

In various embodiments, moving the vascular access device comprises utilizing feedback from a vascular vessel position sensor to ascertain the desired position. For example, the vascular vessel position sensor may include a transducer mounted to the frame of the vascular access device. In various embodiments, the method 290 may further include identifying a first needle of the plurality of needles that has its tip positioned within a lumen of the vascular vessel, as mentioned above with reference to FIG. 7. In various embodiments, the method 290 may also include inserting a guide wire through the first needle, inserting a catheter along the guide wire into the lumen of the vascular vessel of the patient, and/or deploying an occlusion balloon through the catheter and into the lumen of the vascular vessel.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure.

The scope of the disclosure is accordingly to be limited by nothing other than the appended claims, in which reference to an element in the singular is not intended to mean “one and only one” unless explicitly so stated, but rather “one or more.” It is to be understood that unless specifically stated otherwise, references to “a,” “an,” and/or “the” may include one or more than one and that reference to an item in the singular may also include the item in the plural. All ranges and ratio limits disclosed herein may be combined.

Moreover, where a phrase similar to “at least one of A, B, and C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

The steps recited in any of the method or process descriptions may be executed in any order and are not necessarily limited to the order presented. Furthermore, any reference to singular includes plural embodiments, and any reference to more than one component or step may include a singular embodiment or step. Elements and steps in the figures are illustrated for simplicity and clarity and have not necessarily been rendered according to any particular sequence. For example, steps that may be performed concurrently or in different order are illustrated in the figures to help to improve understanding of embodiments of the present disclosure.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure.

Systems, methods and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A vascular access device comprising:
a frame comprising a handle;
a linear actuation assembly mounted to the frame, the linear actuation assembly comprising a translating portion configured to translate relative to the frame; and
a needle retainer coupled to the translating portion, wherein the needle retainer is configured to retain a needle.

2. The vascular access device of claim 1, wherein:
the needle retainer is one needle retainer of a plurality of needle retainers;
the plurality of needle retainers is configured to retain a respective plurality of needles;
the plurality of needle retainers is configured to operably translate as a unit with the translating portion relative to the frame.

3. The vascular access device of claim 1, wherein the linear actuation assembly comprises a track along which the translation portion is configured to move.

4. The vascular access device of claim 3, wherein the frame comprises a bottom surface configured to be in direct contact with a body of a patient, wherein the track is oblique relative to the bottom surface of the frame.

5. The vascular access device of claim 4, wherein the needle retainer is configured to retain the needle substantially parallel to the track.

6. The vascular access device of claim 4, wherein:
the handle is a first handle;
the first handle extends above and is substantially perpendicular to the bottom surface of the frame; and
the frame further comprises a second handle disposed between the track and the bottom surface of the frame.

7. The vascular access device of claim 4, wherein the frame comprises a forward lip portion, wherein the forward lip portion is bent upwards relative to the bottom surface of the frame.

8. The vascular access device of claim 7, wherein an angle between the forward lip portion and a plane of the bottom surface is between 10 degrees and 25 degrees.

9. The vascular access device of claim 1, wherein the linear actuation assembly comprises a rack and gear assembly and an actuation trigger.

10. The vascular access device of claim 1, wherein the frame defines a compartment configured to house a transducer of a vascular vessel position sensor.

11. A vascular access system comprising:
a vascular access device comprising a frame and a needle retainer, wherein the needle retainer is movably mounted to the frame such that the needle retainer is configured to translate relative to the frame; and
a vascular vessel position sensor comprising a transducer mounted to the frame.

12. The vascular access system of claim 11, wherein:
the needle retainer is one needle retainer of a plurality of needle retainers that is configured to translate as a unit relative to the frame; and
the vascular access system further comprises a plurality of needles respectively retained by the plurality of needle retainers.

13. The vascular access system of claim 12, wherein each needle of the plurality of needles comprises at least one of a transparent tube and translucent tube at a back end of the needle.

14. The vascular access system of claim 13, further comprising:
a guide wire configured to be inserted through a first needle of the plurality of needles in response to a tip of the first needle being positioned within a lumen defined by a vascular vessel of a patient;
a catheter configured to be inserted along the guide wire into the lumen of the vascular vessel of the patient; and
an occlusion balloon configured to be deployed through the catheter into the lumen of the vascular vessel of the patient.

15. The vascular access system of claim 11, wherein:
the frame of the vascular access device defines a compartment; and
the transducer of the vascular vessel position sensor is housed within the compartment.

16. A method of accessing a vascular vessel of a patient, the method comprising:
engaging a bottom surface of a frame of a vascular access device against the patient;
moving the vascular access device relative to the patient to position the vascular access device in a desired position in proximity to the vascular vessel of the patient; and
in response to the vascular device being in the desired position, advancing a needle retained by a needle retainer translatably mounted to the frame of the vascular access device.

17. The method of claim 16, wherein:
the needle is one needle of a plurality of needles;
the needle retainer is one needle retainer of a plurality of needle retainers; and
advancing the needle comprises collectively advancing the plurality of needles as a unit into the patient.

18. The method of claim 17, wherein moving the vascular access device comprises utilizing feedback from a vascular vessel position sensor mounted to the frame of the vascular access device to ascertain the desired position.

19. The method of claim 17, further comprising identifying a first needle of the plurality of needles that has its tip positioned within a lumen of the vascular vessel.

20. The method of claim 19, further comprising:
inserting a guide wire through the first needle;
inserting a catheter along the guide wire into the lumen of the vascular vessel of the patient; and
deploying an occlusion balloon through the catheter into the lumen of the vascular vessel of the patient.
